(19) United States
(12) Patent Application Publication
(10) Pub. No.: US 2025/0082929 A1
(43) Pub. Date: Mar. 13, 2025

(54) TRANSVENOUS PHRENIC NERVE STIMULATION SYSTEM

(71) Applicant: ZOLL RESPICARDIA, INC., Minnetonka, MN (US)

(72) Inventors: Randy W. Westlund, St. Paul, MN (US); Mark Gelfand, New York, NY (US)

(21) Appl. No.: 18/957,241

(22) Filed: Nov. 22, 2024

Related U.S. Application Data
(62) Division of application No. 16/525,060, filed on Jul. 29, 2019, now Pat. No. 12,179,014.
Application No. 16/525,060 is a continuation of application No. 12/150,654, filed on Apr. 30, 2008, now Pat. No. 10,406,366.
Application No. 12/150,654 is a continuation-in-part of application No. 11/601,150, filed on Nov. 17, 2006, now Pat. No. 8,244,359.
(60) Provisional application No. 60/926,910, filed on Apr. 30, 2007.
(60) Provisional application No. 60/743,326, filed on Feb. 21, 2006.
(60) Provisional application No. 60/743,062, filed on Dec. 21, 2005.
(60) Provisional application No. 60/737,808, filed on Nov. 18, 2005.

(51) Int. Cl. A61N 1/05; A61N 1/36; A61N 1/368

(52) U.S. Cl. CPC A61N 1/0558; A61N 1/05; A61N 1/0551; A61N 1/3605; A61N 1/3611; A61N 1/3601; A61N 1/3684

(57) ABSTRACT

Examples of the disclosure include methods of implanting stimulation leads.

### CROSS REFERENCE TO RELATED CASES

The present case claims the benefit of and incorporates by reference U.S. Provisional Application 60/926,910 filed Apr. 30, 2007 entitled “Leads for Transvenous Phrenic Stimulation”. The present case also claims the benefit of and incorporates by reference and is a continuation-in-part of U.S. Utility application Ser. No. 11/601,150 filed Nov. 17, 2006 entitled “System and Method to Modulate Phrenic Nerve to Prevent Sleep Apnea”.

### FIELD OF THE INVENTION

The present invention relates generally to a method of implanting a phrenic nerve stimulation lead system and a related phrenic nerve stimulation lead for use with an implanted pulse generator (IPG) for treating a breathing disorder.

### BACKGROUND OF THE INVENTION

Many patients with breathing disorders such as central sleep apnea (CSA) display periods of rapid respiration followed by a relatively long compensatory pause in respiration. The clinical manifestation of the disorder is a period of shallow rapid breathing followed by frank apnea or hypopnea. This pattern repeats episodically and is called Cheyne Stokes Respiration (CSR). Several treatment regimes have been proposed to alleviate CSR, including a technique presented in detail in the utility application incorporated by reference.

Historically, the ability to control respiration via phrenic nerve stimulation is widely known and well reported in the literature. Early work shows the use of phrenic nerve stimulation to treat paralyzed patients to initiate and support respiration. A substantial body of animal research discloses the basic mechanisms for respiration control though stimulation of the phrenic nerve.

Although phrenic nerve stimulation is known in the art there is a continuing need to improve the “leads” devices for accessing and electrically stimulating the phrenic nerve. And there is a continuing need to improve the stimulation methodology.

### SUMMARY OF THE INVENTION

The phrenic nerve stimulation lead device has a flexible elongate lead body with a proximal connector and a distal tip. In use the lead is permanently implanted in a vein near one portion of the phrenic nerve. The lead has physical features and properties important for successful transveous deployment and stimulation of the phrenic nerve from the left pericardiophrenic vein.

The stimulation lead has a distal tip tapered into a “rats tail”. The presence of this extended tapered section will serve to orient and stabilize the lead and the electrodes in the vessel by restricting movement of the lead with respect to the vessel. The additional surface area of the lead provides additional friction and ensures that the vessel and lead do not move relative to each other. One or more and preferably two electrode sites are placed proximal of this distal tip. Each electrode is typically formed as a ring and individually electrically coupled to the proximal connector by internal conductors within the lead.

In one embodiment a guidewire lumen is carried entirely through the lead body and the lumen is concentric with the distal tip at the distal tip. In an alternate embodiment the lead is stiffened by a removable stylet that is inserted into the lead into a stylet lumen.

An optional mechanical stop feature may be included within the lead body to intercept and interact with a finishing guide wire to stabilize the lead during placement.

The lead is acutely repositionable but anticipated foreign-body response will render it permanent in the vessel. The lead may have steroid eluting features to regulate this physiologic process.

The shape of the lead body includes two or more curves, bends or loops near the distal end of the lead. These curves in the lead body lie in two planes and direct the tip at an angle. These features stabilize the lead in a large companion vessel while biasing the distal “rats tail” into a stable position in the smaller target vessel.

The preferred implantation process requires a percutaneous puncture to access the subclavian vein. The implanted pulse generator (IPG) will be implanted in a subcutaneous pocket nearby. A guide catheter having a shaped tip is navigated along the subclavian vein using a guidewire. The catheter and wire pass through the brachiocephalic vein in to the ostium of the left pericardiophrenic vein. Normal contrast venography techniques are used to illuminate and access this location. The guidewire is inserted several centimeters into the left pericardiophrenic vein and the mouth of the guide catheter is passed into the ostium of the left pericardiophrenic vein. Next the stimulation lead is delivered to a target location through the guide catheter over the guidewire alone or with the use of a stylet. When the electrodes are well positioned near the phrenic nerve target location the stylet or guidewire is removed and the optional stabilizing or finishing guidewire wire is exchanged and inserted into the lead body. Relative traction between the finishing guide wire and the guide sheath allows for the smooth removal of the guide catheter without dislodging the lead. In essence the “rats tail” remains biased and stationary in the left pericardiophrenic vein as the lead “relaxes” and assumes its natural low mechanical energy state while the guide catheter is removed. Withdrawal of the finishing wire if used or the guidewire or stylet activates the complementary shaped curves of the lead. As the curves bend and unfurl into contact with the larger brachiocephalic vein the most distal tip of the of the lead in the smaller vessel becomes stabilized. Next the proximal connector of the lead is coupled to the IPG. The IPG provides stimulation that completes the implantation method and the method of therapy.

### BRIEF DESCRIPTION OF THE DRAWINGS

Identical reference numerals indicate identical features throughout the figures of the drawing, wherein:

FIG. 1 depicts the implanted system;

FIG. 2 depicts the lead device;

FIG. 3 depicts a step in method used to implant the device;

FIG. 4 depicts the lead in position in the target vessel with the electrodes positioned at the target location.

### DETAILED DESCRIPTION OF THE INVENTION

### Stimulation Regime

The applicant has incorporated a utility patent reference that discloses in detail a medical device (IPG) that can detect and treat CSR and other forms of breathing disorders by the transveous electrical stimulation of the phrenic nerve. For purposes of this disclosure it is sufficient to understand that the disclosed technique uses electrical stimulation of one phrenic nerve to arrest or still the motion of one hemidiaphram of the patient. This process lowers the observed breathing rate post therapy and over time drives the blood gases to an improved state of oxygen saturation and carbon dioxide elimination.

The implanted pulse generator (IPG) 12 has the ability to detect the respiration process in real time. Preferably impedance plesthmography is used to detect both the rate of respiration and the turning points within a single breath. It is anticipated that the companion IPG 12 includes an impedance plethysmograph that emits minute electrical pulses between electrodes on a measurement lead system (not shown in the present figures). These impedance signals are used to measure the volume of the lung and rate of change of volume of the lung. The phrenic nerve stimulation therapy is provided after the start of a breath but before the natural end of the breath. The magnitude of the stimulation is sufficient to arrest the motion of the diaphragm. By essentially stopping the breathing for a moment the overall duration of the breath is extended. This breath hold process lowers the observed rate of breathing of at least one lung.

This stimulation therapy may be supplied to each breath for a series of breaths or on a less frequent basis. The stimulation may be supplied in response to a detected episode of CSR or it may be provided to prevent progression to CSR. For example, stimulation may be initiated upon the detected occurrence of CSR. Alternatively an activity sensor may report that the patient is supine and at rest and this set of criteria may be necessary and sufficient to invoke therapeutic stimulation. Regardless of the specific intervention criteria, the IPG will delivery the appropriate amount of energy to still the breath, via the transvenous stimulation lead.

### System Architecture

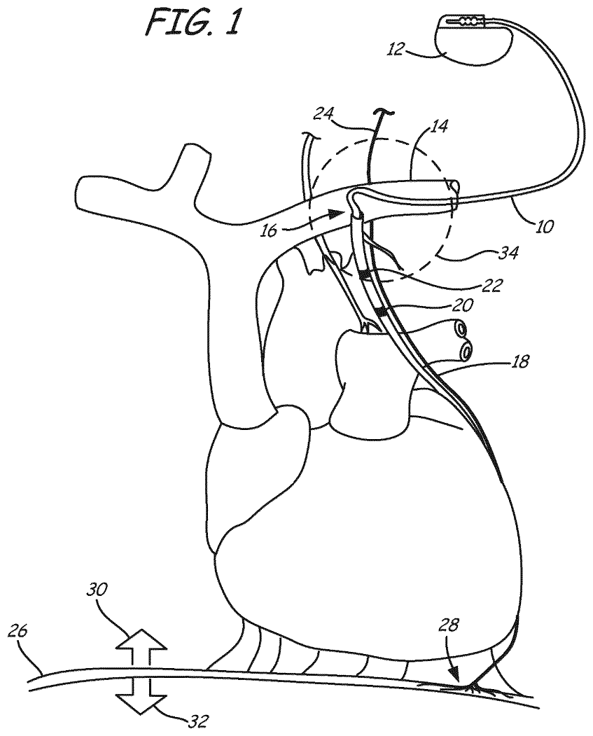

FIG. 1 depicts the overall architecture and context of the therapy where the lead 10 coupled to an implanted pulse generator 12. The lead is inserted through the brachiocephalic vein 14 into the ostium 16 of the left pericardiophrenic vein 18. In this chronic condition the two electrodes 20 and 22 can deliver electrical stimulation to the phrenic nerve 24, which courses parallel to the vein 18 in this target vessel at this target location. This half of the branching phrenic nerve 24 terminates in the hemidiaphram. 26 as indicated by the innervations depicted at location 28. The nerve innervations excite the muscles of the hemidiaphram which move downward as indicated by motion arrows 32 to produce inspiration followed by upward motion as indicated by motion arrow 30 to produce expiration. Together the motion arrows represent rhythmic respiration. For purposes of orientation other familiar anatomic structures are seen but not labeled in the figure.

In summary after implantation the lead 10 system delivers electrical stimulation to the phrenic nerve to arrest diaphragm 26 motion by the delivery of electrical energy after the onset of inspiration indicated by motion arrow 32 in the figure and the electrical energy delivered is sufficient to pause that diaphragm motion. In this fashion the lead 10 system and the IPG 12 are used for stimulating the phrenic nerve 24 of a patient to treat defects in respiration.

### Stimulation Lead

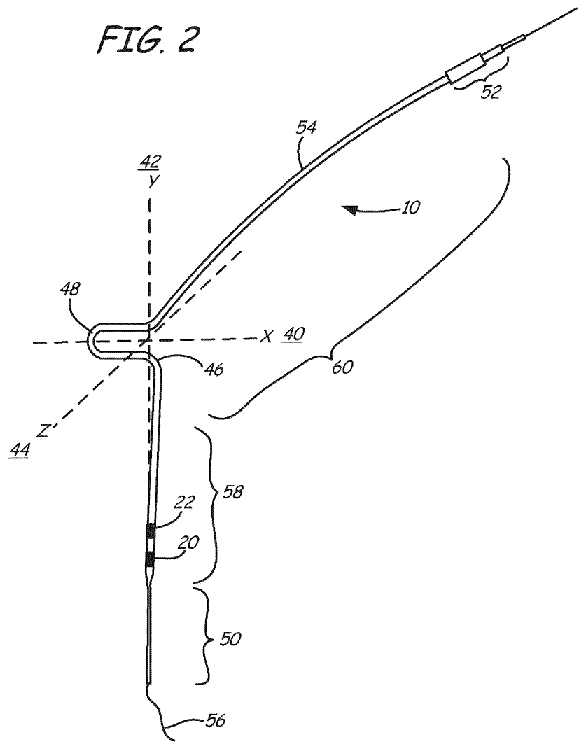

FIG. 2 depicts the lead system 10 in isolation. The lead is best considered by dividing it up into various segments. The most distal segment 50 takes the form of a narrow taper. This “rats tail” is coupled to a stimulation segment 58 that includes a first electrode 22 and a second electrode 20. Although the preferred exemplary embodiment shows two electrodes, other numbers of electrodes are operable and desirable in some situations. The electrode placement on the leads seen in the figures is desirable because to the extent possible it is desired to have the stimulation current path transect the longitudinal fibers of the phrenic nerve 24 at the target location. It has been determined experimentally that this orientation reduces thresholds for stimulation of the nerve.

Next, an intermediate shaped segment is shown at numeral 60. The shaped segment includes two or more bends or loops or curves. The bend curve 48 lies in the XZ plane in the figure. The bend curve 46 rises out of the XZ plane in the Y direction. Preferably the axis of the distal segment 50 makes an included angle of about 15 degrees with respect to the XZ plane.

The most proximal segment includes a connector pin assembly 52 that allows conductors within the lead to communicate with the two electrodes. For clarity the conductors are not shown. The construction of the conductors is well known in this art and need not be shown in detail. Preferably and overall the elongate portion 54 of the shaped segment 60 and the distal segment 50 are not coplanar and the major axis of the elongate portion 54 and major axis of the distal segment 50 are not coaxial. These geometric constraints place the elongate segment 54 and the distal segment 50 in separate planes and the major axes of these sections of the lead are not collinear.

FIG. 2 shows the lead 10 in isolation in its low stress state. The lead has a natural neutral bias in the figure and the lead structure and shape gives rise to a friction zone caused by a in-plane deflection of the lead around first primary radius of bend curve 48 and a secondary radius of bend curve 46. In use the curvilinear structures will permit the stable positioning of the lead body in the brachiocephalic vein and permit entry of the stimulation segment into the ostium of the left pericardiophrenic vein and stabilize the electrodes at the target location.

The lead may also have a through lumen to accept a guide wire 56 as depicted in the figure passing into the connector 52 pin and traveling beyond the distal tip and emerging at reference numeral 56. As an alternative, a stylet lumen may be located within the lead to permit the use of a stylet to stiffen the lead. It may also be desirable to have a mechanical stop in the stylet or guide wire lumen to accept a “finishing wire”. This optional finishing wire can be used to supply a force to the lead to keep it in position as the guide catheter is removed. In general the finishing wire is of slightly larger diameter and it bottoms out at a location near but still proximal of the electrode and shaped segments of the lead. Pulling on the guide catheter while pushing on the finishing wire at the same time prevents the lead to guide catheter friction from dislodging or moving the electrodes from their preferred location.

### Method of Implantation

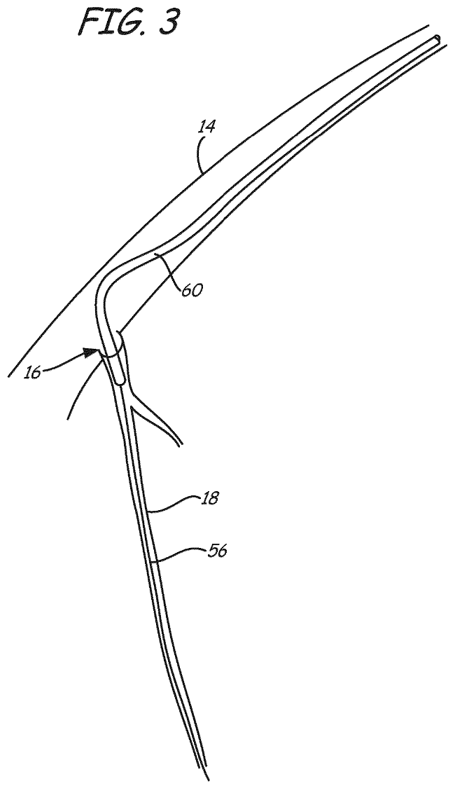

FIG. 3 is an enlarged portion of part of FIG. 1 designated by numeral 34 on FIG. 1. The brachiocephalic vein 14 and the branching left pericardiophrenic vein 18 are shown in isolation. The figure illustrates a method of implanting a lead in a small vein 18 that branches off from a large vein 14. The process begins with a “percutaneous stick” to access a large vein connecting to the brachiocephalic vein. A sharp hollow needle trocar enters the vein and a guidewire is advanced through the trocar into the vessel. The trocar is withdrawn over the wire and replaced with a sheath which is passed into the vessel. Next a guide wire and guide catheter of the type having a distal curve are navigated to the ostium 16 of the left pericardiophrenic vein. The curved tip for the guiding catheter is introduced in to this small vein, Venographic imaging technologies such as contrast injection and biplane fluoroscopy are used to locate the ostium 16. With both the guide wire and guide catheter in the small vein the lead 10 may be passed over the wire into the vein. Under contrast imaging and temporary stimulation the best spot for activating the phrenic nerve is located. This defines the target location. Next the guidewire and guiding catheter are carefully removed while holding the lead in position with the optional finishing wire if present. The finishing wire if used compensates for the friction between the guide catheter 60 and the lead 10 which would otherwise causes the guide catheter to tend to drag the lead out of position as the guide catheter is removed.

### Lead Interactions

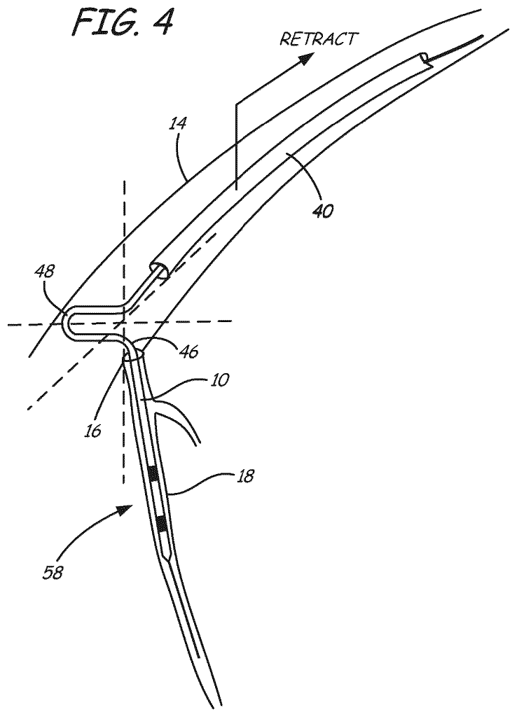

Turning to FIG. 4 once again there is a shown an enlarged section of FIG. 1 identified in that figure by reference numeral 34. FIG. 4 shows the lead 10 delivered through a guide sheath 40. A stylet or guidewire (G W) may be inserted into the lead 10 to straighten and stiffen the structure. Once the lead enters the target vessel the stylet may be removed and the lead adopts its low stress state in the vessel. In this figure the biasing mechanism 46 is shown in contact with the walls of the vessel 18. The bend 48 lies in a single plane in contact with the wall of vessel 14. The bend or shape 46 exerts a force against the ostium 16 to help anchor the electrodes and stimulation segment in the smaller target vessel 18.

Steroid eluting features may be provided on portions of the lead system to reduce inflammation associated with the placement of the leads. Other coatings maybe used to enhance or reduce friction to help stabilize the lead.

1-8. (canceled)

9. A method of implanting a stimulation lead at a target venous location in a patient, the method comprising:
maneuvering the stimulation lead through a brachiocephalic vein and into an ostium of a left pericardiophrenic vein that branches off of the brachiocephalic vein, the stimulation lead having a proximal end and a distal end, and
positioning the distal end of the stimulation lead near the target venous location within the left pericardiophrenic vein, wherein the target venous location is proximate to a left phrenic nerve,
wherein the stimulation lead has at least one shaped section comprising a plurality of bend curves, wherein at least one bend curve of the plurality of bend curves is shaped to deflect around a radius such that, when the distal end of the stimulation lead is positioned near the target venous location, a bias force exerted against a wall of the left pericardiophrenic vein stabilizes the stimulation lead in the left pericardiophrenic vein.

10. The method of claim 9, wherein the stimulation lead comprises a plurality of electrodes located along the stimulation lead near the distal end and configured to stimulate the left phrenic nerve.

11. The method of claim 10, wherein all bend curves of the plurality of bend curves are free of any electrodes of the plurality of electrodes.

12. The method of claim 10, comprising positioning the distal end of the stimulation lead near the target venous location so that the plurality of electrodes is in contact with the wall of the left pericardiophrenic vein proximate to the left phrenic nerve.

13. The method of claim 9, wherein the proximal end includes a proximal electrical connector, the method comprising coupling the proximal electrical connector to an implanted IPG such that the implanted IPG and the stimulation lead are coupled.

14. The method of claim 9, comprising accessing a subclavian vein connected to the brachiocephalic vein with a percutaneous stick and introducing a hollow needle trocar into the subclavian vein.

15. The method of claim 9, comprising maneuvering a guidewire through the brachiocephalic vein and into the ostium of the left pericardiophrenic vein that branches off of the brachiocephalic vein.

16. The method of claim 15, comprising passing the stimulation lead over the guidewire to maneuver the stimulation lead through the brachiocephalic vein and into the ostium of the left pericardiophrenic vein that branches off of the brachiocephalic vein.

17. The method of claim 16, comprising passing the stimulation lead over the guidewire such that the guidewire passes into a proximal electrical connector, passes through a lumen in the stimulation lead, and extends out of the distal end of the stimulation lead.

18. The method of claim 15, comprising inserting the guidewire into the stimulation lead to maneuver the guidewire through the brachiocephalic vein, straightening the stimulation lead with the insertion of the guidewire, and maneuvering the stimulation lead straightened with the guidewire into the ostium of the left pericardiophrenic vein that branches off of the brachiocephalic vein.

19. The method of claim 15, comprising removing the guidewire from the patient.

20. The method of claim 19, comprising removing the guidewire from the patient while holding the stimulation lead in position with a finishing wire.

21. The method of claim 9, comprising inserting a stylet into the stimulation lead to maneuver the stimulation lead through the brachiocephalic vein, straightening the stimulation lead with the insertion of the stylet, and maneuvering the stimulation lead straightened with the stylet into the ostium of the left pericardiophrenic vein that branches off of the brachiocephalic vein.

22. The method of claim 9, comprising maneuvering a guide catheter having a distal curve through the brachiocephalic vein and into the ostium of the left pericardiophrenic vein that branches off of the brachiocephalic vein.

23. The method of claim 22, comprising passing the stimulation lead through the guide catheter having the distal curve to maneuver the stimulation lead through the brachiocephalic vein and into the ostium of the left pericardiophrenic vein.

24. The method of claim 22, comprising removing the guide catheter having the distal curve from the patient.

25. The method of claim 24 comprising removing the guide catheter having the distal curve from the patient while holding the stimulation lead in position with a finishing wire.

26. The method of claim 9, comprising passing the stimulation lead through a guide sheath to maneuver the stimulation lead through the brachiocephalic vein and into the ostium of the left pericardiophrenic vein.

27. The method of claim 9, comprising locating the target venous location based on venographic imaging.

28. The method of claim 27, wherein the venographic imaging comprises contrast injection and biplane fluoroscopy.

29. The method of claim 9, comprising locating the target venous location based on temporary stimulation of the left phrenic nerve proximate the target venous location.

30. A method of implanting a stimulation lead at a target venous location in a patient in a small vein branched off from a large vein, the method comprising:
maneuvering a guide catheter having a distal curve through a brachiocephalic vein and into an ostium of a left pericardiophrenic vein that branches off of the brachiocephalic vein,
maneuvering a guidewire through the brachiocephalic vein and into the ostium of the left pericardiophrenic vein that branches off of the brachiocephalic vein,
passing the stimulation lead over the guidewire and through the guide catheter having the distal curve to maneuver the stimulation lead through the brachiocephalic vein and into the ostium of the left pericardiophrenic vein, the stimulation lead having a proximal end and a distal end, and
positioning the distal end of the stimulation lead near the target venous location within the left pericardiophrenic vein, wherein the target venous location is proximate to a left phrenic nerve,
wherein the stimulation lead has at least one shaped section comprising a plurality of bend curves, wherein at least one bend curve of the plurality of bend curves is shaped to deflect around a radius such that, when the distal end of the stimulation lead is positioned near the target venous location, a bias force exerted against a wall of the left pericardiophrenic vein stabilizes the stimulation lead in the left pericardiophrenic vein.

31. The method of claim 30, wherein the stimulation lead comprises a plurality of electrodes located along the stimulation lead near the distal end and configured to stimulate the left phrenic nerve.

32. The method of claim 31, wherein all bend curves of the plurality of bend curves are free of any electrodes of the plurality of electrodes.

33. The method of claim 31, comprising positioning the distal end of the stimulation lead near the target venous location so that the plurality of electrodes is in contact with the wall of the left pericardiophrenic vein proximate to the left phrenic nerve.

34. The method of claim 30, wherein the proximal end includes a proximal electrical connector, the method comprising coupling the proximal electrical connector to an implanted IPG such that the implanted IPG and the stimulation lead are coupled.

35. The method of claim 30, comprising accessing a subclavian vein connected to the brachiocephalic vein with a percutaneous stick and introducing a hollow needle trocar into the subclavian vein.

36. The method of claim 30, comprising removing the guidewire and the guide catheter from the patient.

37. The method of claim 36, comprising removing the guidewire and the guide catheter from the patient while holding the stimulation lead in position with a finishing wire.

38. The method of claim 30, comprising locating the target venous location based on one or more of temporary stimulation of the left phrenic nerve proximate the target venous location or venographic imaging comprising contrast injection and biplane fluoroscopy.
